(19) United States
(12) Patent Application Publication
(10) Pub. No.: US 2022/0061726 A1
(43) Pub. Date: Mar. 3, 2022

(54) METHOD FOR CHARACTERIZING, DETECTING, AND MONITORING ADHD

(71) Applicant: Sentio Solutions, Inc., San Francisco, CA (US)

(72) Inventors: Georgios Eleftheriou, Santa Clara, CA (US); Panagiotis Fatouros, Athens (GR); Charalampos Tsirmpas, Athens (GR)

(21) Appl. No.: 17/461,494

(22) Filed: Aug. 30, 2021

Related U.S. Application Data
(60) Provisional application No. 63/072,079, filed on Aug. 28, 2020.

(51) Int. Cl. A61B 5/16; A61B 5/316; G06K 9/00

(52) U.S. Cl. CPC A61B 5/168; A61B 5/316; A61B 5/162; A61B 5/165; G06K 9/00892

(57) ABSTRACT

One variation of a method for characterizing ADHD includes: accessing results of a cognitive evaluation executed by a user; accessing a timeseries of biosignal data collected via sensors worn by the user during execution of the cognitive evaluation; accessing a global model linking results of the cognitive evaluation and biosignal data to cognitive states of users diagnosed with ADHD; and interpreting a confidence score, representing probability of an ADHD diagnosis, based on results of the cognitive evaluation, the timeseries of biosignal data, and the global model. The method further includes, in response to the confidence score exceeding a threshold confidence: selecting a treatment pathway for the user based on results of the cognitive evaluation and the timeseries of biosignal data; and deriving an ADHD model for the user linking biosignal data to cognitive states based on results of the cognitive evaluation, the timeseries of biosignal data, and the global model.

### CROSS-REFERENCE TO RELATED APPLICATIONS

This application claims the benefit of U.S. Provisional Application No. 63/072,079, filed on 28 Aug. 2020, which is incorporated in its entirety by this reference.

### TECHNICAL FIELD

This invention relates generally to the field of biosensors and more specifically to a new and useful method for detecting and monitoring ADHD biomarkers in the field of biosensors.

### BRIEF DESCRIPTION OF THE FIGURES

FIG. 1 is a flowchart representation of a method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

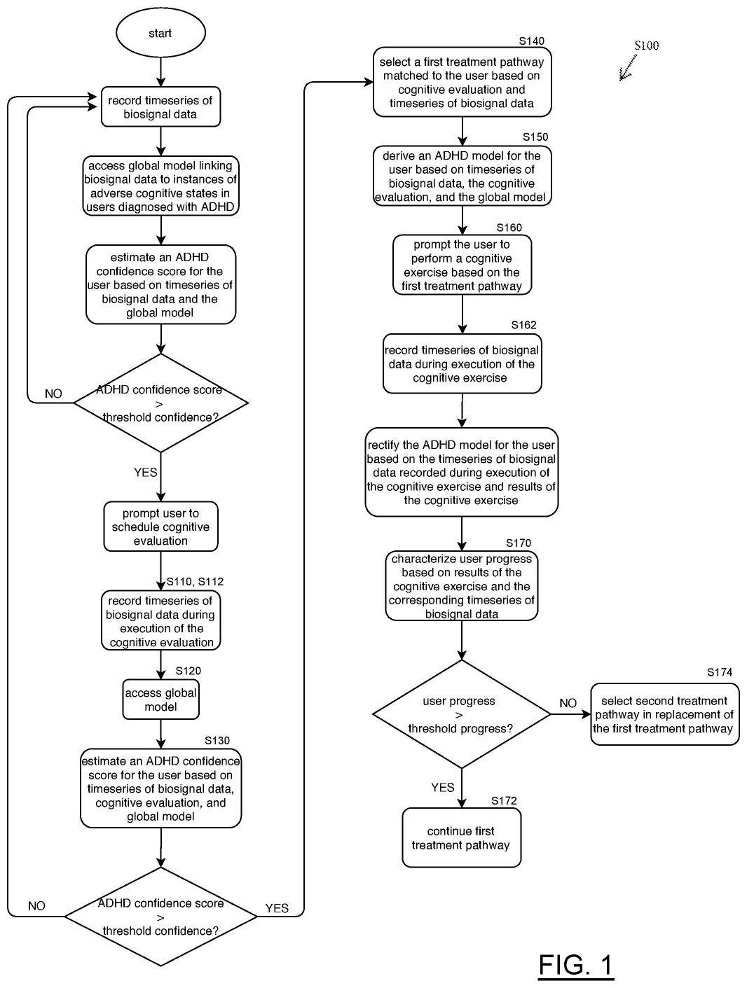

As shown in FIG. 1, a method S100 includes, during a first period of time: accessing a series of results of a cognitive evaluation executed by the user during a test period in Block Silo; accessing a first timeseries of biosignal data collected via a set of sensors integrated into a wearable device worn by the user during the test period in Block S112; accessing a global model linking results of the cognitive evaluation and biosignal data to cognitive states of users diagnosed with ADHD in Block S120; interpreting a first confidence score for the user based on the series of results of the cognitive evaluation, the first timeseries of biosignal data, and the ADHD diagnostic model in Block S130, the first confidence score representing a probability of a positive ADHD diagnosis for the user; and, in response to the first confidence score exceeding a threshold confidence, selecting a first treatment pathway matched to the user based on the series of results of the cognitive evaluation and the timeseries of biosignal data in Block S140; and deriving an ADHD model for the user linking biosignal data recorded for the user to instances of cognitive states exhibited by the user based on the series of results of the cognitive evaluation, the first timeseries of biosignal data, and the global model in Block S150.

The method S100 further includes, during a second period of time: prompting the user to complete a cognitive exercise based on the first treatment pathway selected for the user in Block S160; recording a second timeseries of biosignal data during execution of the cognitive exercise in Block S162; characterizing user progress based on results of the cognitive exercise and the second timeseries of biosignal data in Block S170; in response to user progress exceeding a threshold progress, confirming selection of the first treatment pathway for the user in Block S72; and, in response to user progress falling below the threshold progress, selecting a second treatment pathway in replacement of the first treatment pathway in Block S174.

In one variation, the method S100 further includes: estimating a second confidence score for the user based on results of the cognitive exercise, the second timeseries of biosignal data, and the ADHD model generated for the user; and, in response to the second confidence score exceeding the threshold confidence, affirming the positive ADHD diagnosis for the user.

### 2. Applications

Generally, Blocks of the method S100 can be executed by a companion application executing on a mobile device in cooperation with a wearable device worn by a user and/or by a remote computer system (hereinafter the “system”) to detect instances of adverse cognitive states related to attention-deficit hyperactivity disorder (hereinafter “ADHD”) based on: correlations between physiological biosignal data (e.g., heart-rate, heart-rate variability, skin temperature, skin moisture, electrodermal activity, etc.) for the user; and interference in various executive functions (e.g., attentional control, cognitive inhibition, working memory) of the user, which may be caused by ADHD. Based on these instances of adverse cognitive states, the system can interpret an ADHD diagnosis (e.g., positive or negative) for the user and suggest treatment pathways matched to this ADHD diagnosis and this user.

In one implementation, the system can onboard a user: seeking a better understanding of her ADHD and better ADHD coping strategies; seeking an ADHD diagnosis for the first time based on past patterns of problems with productivity and functioning in daily life, such as patterns of problems surfaced by the companion application after a period of use; or unknowingly suffering from ADHD but prompted to investigate an ADHD diagnosis by the system based on correlations between biosignal data for the user—collected over a period of use by the user—and users previously diagnosed with ADHD.

In particular, the companion application can prompt a user to complete a cognitive evaluation (e.g., a “Stroop test”) configured to evaluate interference in executive functions (e.g., attentional control, cognitive inhibition, working memory) of the user, which may return indicators of ADHD. During execution of the cognitive evaluation, the wearable device can record a timeseries of physiological biosignal data of the user via a suite of integrated sensors. The wearable device can offload the timeseries of physiological biosignal data to the remote computer system—such as via the mobile device in real-time or in intermittent data packets—and the remote computer system can then access a global ADHD model associating results of the cognitive evaluation, biosignal data, and instances of adverse cognitive states (e.g., reduced attentional control, reduced cognitive inhibition, limited working memory) caused by ADHD. The remote computer system can therefore leverage the user's cognitive evaluation results, the concurrent timeseries of biosignal data, and the global ADHD model to interpret whether the user exhibits instances of adverse cognitive states during execution of the cognitive evaluation and thus interpret whether the user exhibits cognitive behaviors associated with ADHD.

By leveraging biosignal data in combination with results of the cognitive evaluation, the system can extract deeper insights into the ADHD diagnosis of the user. For example, rather than interpreting a binary ADHD diagnosis (e.g., a positive or negative diagnosis) for the user, the system can interpret ADHD diagnoses along a spectrum and therefore define an ADHD profile specific to the user. This ADHD profile can define: a confidence indicating a probability of a positive ADHD diagnosis; a type of ADHD (e.g., attention-deficit, hyperactive, or combination); and/or an intensity (e.g., low, medium, or high) of ADHD exhibited by the user. The system can then update this ADHD profile for the user over time as the system receives or captures additional user information (e.g., biosignal data, cognitive testing, user feedback, third-party feedback).

Further, the system can leverage the results of the cognitive evaluation and the timeseries of biosignal data collected during execution of the cognitive evaluation to derive a user-specific ADHD model linking physiological biosignals with instances of adverse cognitive states for the user. For example, the system can: access a series of results of the cognitive evaluation; and transform the series of results of the cognitive evaluation into timestamped instances (and magnitudes) of the adverse cognitive state exhibited by the user while completing the cognitive evaluation (e.g., based on a correlation between results of the cognitive evaluation and cognitive state). The system can then: access the timeseries of biosignal data recorded during execution of the cognitive evaluation; synchronize the timeseries of biosignal data and timestamped instances of the adverse cognitive state; and implement regression, machine learning, deep learning, and/or other techniques to derive correlations between biosignal data and instances of the adverse cognitive state for the user.

The system can implement similar techniques to derive correlations between biosignal data and instances of other cognitive states exhibited by the user and compile each of these correlations into a user-specific ADHD model configured to detect magnitudes, changes, and/or patterns of cognitive states for the user. Thus, the system can learn a set of biomarkers (or “ADHD biomarkers”) indicative of the adverse cognitive state when detected in the biosignal data for this user.

The system can continue monitoring cognitive states of the user over time to (regularly, continuously) refine the user-specific ADHD model and implement this module to identify intervention need and to serve intervention activities to the user in near real-time, thus enabling the user to monitor and mitigate instances of adverse cognitive events caused by ADHD. For example, the system can periodically prompt the user to complete cognitive exercises—while recording biosignal data of the user—configured to monitor the user's progression; and then confirm, refine, or reject the user-specific ADHD model and the user's ADHD diagnosis based on results of these subsequent cognitive exercises. Further, by learning a set of ADHD biomarker signals (e.g., ADHD biomarker expression) specific to the user and monitoring the user's biosignals on a regular basis (e.g., continuously, hourly, daily), the system can detect and alert (e.g., via the wearable device) the user of adverse cognitive events as they occur in real or near-real time, enabling the user to extract insights into these events as well as react accordingly.

Additionally, the mobile device or companion application can load a particular intervention activity (e.g., a breathing activity, a journaling activity) matched to the cognitive state of the user; and prompt the user to complete the particular intervention activity via the mobile device when the wearable device detects an instance of the adverse cognitive state. By prompting the user (or “intervening”) during an adverse cognitive event, the system can enable the user: to focus on less engaging tasks and/or an intervention activity; and to isolate and record a circumstance that triggered the adverse cognitive event. Concurrently, by prompting the user during instances of a high performance and functionality zone (e.g., linked to improved performance, operation, cognitive behavior of the user), the system can enable the user: to attempt more engaging tasks during an instance of the high performance and functionality zone; and to isolate and record a circumstance that triggered this instance of the high performance and functionality zone and thus guide the user toward developing successful coping strategies.

Therefore, the system can leverage biosignal data of the user—in combination with cognitive evaluations performed by the user—to diagnose ADHD, detect instances of adverse cognitive states caused by ADHD, and suggest treatment pathways including intervention techniques matched to the user for mitigation of ADHD.

### 3. Wearable Device & Companion Application

Blocks of the method S100 can be executed by a companion application executing on a mobile device in cooperation with a wearable device worn by a user and a remote computer system (or “the system”). The wearable device can record a timeseries of physiological biosignal data of the user via a set of sensors integrated into the wearable device. The wearable device can offload the timeseries of physiological biosignal data to the mobile device—such as in real-time or in intermittent data packets—and the companion application can return these data to the remote computer system.

During a calibration period, the system can: load a companion application onto a user's mobile device; wirelessly (or via wired connection) connect the mobile device to the wearable device; run evaluations to confirm the functionality and accuracy of the set of sensors (e.g., an electrodermal activity sensor, a heart rate sensor, a skin temperature sensor, an inertial measurement unit (or “IMU”), an ambient humidity sensor, and an ambient temperature sensor) integrated into the wearable device; and prompt the user to enter demographic data (e.g., age, sex, education level, income level, marital status, occupation, weight, etc.) to generate a user profile. Generally, the companion application can—upon loading onto the mobile device—prompt the user to enter demographic user data to predetermine expected ranges of biosignal data for the user (e.g., a higher average skin temperature for a male user, a lower average skin temperature for a user of below average weight, a higher average skin conductance for a user in a high stress job, etc.).

Generally, the wearable device can access a set of sensors (e.g., an electrodermal activity sensor (or “EDA” sensor), a heart rate or photoplethysmogram sensor (or “PPG” sensor), a skin temperature sensor or (“STE” sensor), an inertial measurement unit (hereinafter “IMU”), an ambient humidity sensor, and an ambient temperature sensor) and record biosignal data at each sensor at a series of time increments.

In one implementation, the system can access: the electrodermal activity sensor to record the skin conductance of the user; the heart rate sensor to record the pulse of the user; the IMU to record the motion of the user; the skin temperature sensor to record the user's skin temperature; the ambient humidity sensor to record the relative humidity of the air around the user; and the ambient temperature sensor to record the relative heat of the air around the user.

Similarly, the wearable device can record a baseline resting heart rate, a baseline skin conductance, and a baseline level of activity for the user, and store all the baseline data locally on the wearable device or on the remote computer system as part of a user profile.

In one implementation, the wearable device can sample biosignal data intermittently (e.g., one a five-second interval) to reduce power consumption and minimize data files. In another implementation, the wearable device can selectively choose times to record biosignal data continuously instead of intermittently (e.g., if the system detects the user is trending toward an instance of an adverse cognitive state). After recording the biosignal data at the wearable device, the wearable device can transmit the biosignal data to the mobile device for storage in the user's profile, such as locally on the mobile device and/or remotely in a remote database.

In one implementation, the wearable device records physiological biosignal data of the user (e.g., skin moisture, skin temperature, and heart rate variability) while concurrently recording ambient environmental data (e.g., humidity, ambient temperature) and other related data (e.g., the motion of the user or motion of the user's mode of transportation). For example, the wearable device can: access the electrodermal activity sensor to detect the user's current skin moisture data; identify that the user's current skin moisture data is above a normal threshold; access the ambient humidity sensor to detect an ambient humidity level; identify that the ambient humidity sensor is above the normal threshold; identify that the ambient humidity level is affecting the skin moisture data; and calculate a real skin moisture level based on the ambient humidity level. Therefore, the system can identify environmental situations that can affect the biosignal data of the user (e.g., washing her hands, running, etc.).

Furthermore, the wearable device can: access the set of sensors integrated into the wearable device worn by the user to acquire a first set of physiological biosignal data; and transmit the first set of physiological biosignal data to the mobile device. The companion application—executing on the user's mobile device—can then validate the first set of physiological biosignal data with generic baseline physiological biosignal data from a generic user profile (e.g., a profile defining a range of standard resting heart rates for a generic user, a range of normal skin temperatures, etc.), or from additional sources (e.g. confirming the ambient humidity recorded by the wearable device with the ambient humidity recorded by a third-party weather service, the time of day with the internal clock on the mobile device, etc.). For example, the wearable device can: record a particular physiological biosignal for the user (e.g., a resting heart rate); access a generic user profile including an acceptable (or expected) range for the particular biosignal (e.g., a resting heart rate between 60-100 beats per minute or “bpm”); and—if the biosignal data is within an acceptable range (e.g., 65 bpm)—store user biosignal data as a normal baseline for the user. Conversely, if the physiological biosignal data is not within the acceptable range (e.g., a resting heart rate of 40 bpm), the system can run diagnostics on the sensor or prompt the user to confirm the wearable device is on (or properly installed). The system can also prompt the user to override data that is out of the acceptable range (e.g., a marathon runner with a resting heart rate of 40 bpm can manually validate her resting heart rate.)

In another implementation, the companion application can: prompt the user to engage in a series of activities (e.g., sitting, walking, holding her breath, etc.); record a series of biosignal data via the set of sensors integrated into the wearable device; label the biosignal data with the associated activity; and store the labeled biosignal data in a user profile to enable the system to eliminate false positives triggered by normal activities.

The method S100 is described as executed by and/or in conjunction with a wearable device. However, the method S100 can be executed by and/or in conjunction with any sensor(s) configured to record physiological biosignal data of the user.

### 4. Onboarding/Diagnostics

In one implementation, after successful calibration of the wearable device and generation of the user profile, the system can instruct a new user to complete a cognitive evaluation (e.g., a Stroop Test)—configured to detect instances of cognitive states related to ADHD—in response to a request by the user. In this implementation, the system can leverage results of the cognitive evaluation and corresponding timeseries of biosignal data to interpret whether an ADHD diagnosis is appropriate for this new user.

In another implementation, the system can prompt an established user to complete a cognitive evaluation—configured to detect instances of cognitive states related to ADHD—in response to detecting a particular trend in biosignal data recorded for the user linked to ADHD. In this implementation, the system can leverage results of the cognitive evaluation to determine whether this particular trend—if observed during the cognitive evaluation—is linked to the user's performance (e.g., the results) for the cognitive evaluation. The system can therefore affirm or reject an ADHD diagnosis for this user. For example, a user may initially purchase an instance of the wearable device to monitor her emotions for management of a mood disorder (e.g., stress, anxiety, depression) as described in U.S. patent application Ser. No. 16/460,105, filed on 2 Jul. 2019, which is incorporated in its entirety by this reference. The system can collect a timeseries of biosignal data for this user over an initial period of time. The system can then check this timeseries of biosignal data for abnormalities (e.g., irregular patterns and/or fluctuations in biosignal data) based on a generic biosignal model representing biosignal data of healthy users (e.g., users not diagnosed with ADHD). In response to detecting a first abnormality linked to ADHD in the timeseries of biosignal data for this user, the system can: access the global ADHD model including biosignal data of users diagnosed with ADHD; characterize a correlation between the first timeseries of biosignal data and biosignal data of users diagnosed with ADHD based on the global ADHD model (e.g., via regression, machine learning, deep learning, and/or other techniques to derive links or correlations); and, in response to the correlation exceeding a threshold correlation, flag this user as a candidate for an ADHD diagnosis and/or prompt the user to complete a cognitive evaluation configured to diagnose ADHD. Additionally and/or alternatively, in response to the correlation exceeding the threshold correlation, the system can prompt the user to consult a mental health care provider (e.g., a psychiatrist).

In yet another implementation, the system can prompt a new or established user, previously diagnosed with ADHD, to complete a cognitive evaluation configured to detect instances of cognitive states related to ADHD. The system can then leverage this confirmed ADHD diagnosis, results of the cognitive evaluation, and a timeseries of biosignal data recorded during execution of the cognitive evaluation to develop a more robust diagnosis for this user as well as suggest treatment strategies to this user based on the diagnosis.

The system can therefore leverage biosignal data to: interpret whether a user may be a candidate for an ADHD diagnosis and suggest further evaluation to the user; interpret whether an ADHD diagnosis is appropriate for a user; and enable a user diagnosed with ADHD to more deeply understand her diagnosis as well as better manage the negative effects of ADHD.

### 4.1 Cognitive Evaluation+Biosignal Data

The system can prompt the user to schedule and/or execute a cognitive evaluation (e.g., a Stroop test) configured to interpret whether an ADHD diagnosis is appropriate for the user. During execution of the cognitive evaluation, the system can collect and record a timeseries of biosignal data via the set of sensors integrated into the wearable device worn by the user. The system can then leverage the results of the cognitive evaluation and the timeseries of biosignal data to interpret whether an ADHD diagnosis is appropriate for the user based on historical biosignal data of other users diagnosed with ADHD.

By recording biosignal data concurrently with execution of the cognitive evaluation, the system can link portions of the cognitive evaluation to segments of the timeseries of biosignal data to extract further insights regarding a type and severity of ADHD for the user. For example, the system can: link a first segment of the timeseries of biosignal data to a first portion of the cognitive evaluation (e.g., a first set of exercises on the cognitive evaluation) configured to evaluate attentional control of the user; link a second segment of the timeseries of biosignal data to a second portion of the cognitive evaluation (e.g., a second set of exercises on the cognitive evaluation) configured to evaluate cognitive inhibition of the user; and link a third segment of the timeseries of biosignal data to a third portion of the cognitive evaluation (e.g., a third set of exercises on the cognitive evaluation) configured to evaluate working memory of the user. Therefore, the system can match performance of the user on the cognitive evaluation to particular areas of the timeseries of biosignal data and thus delineate a set of ADHD biomarkers in the timeseries of biosignal data corresponding to portions of the cognitive evaluation in which the user's results indicate a potential ADHD diagnosis.

### 4.2 ADHD Profile

The system can leverage results of the cognitive evaluation and biosignal data collected during the cognitive evaluation to generate an ADHD profile unique to the user.

In one implementation, the system can leverage these cognitive evaluation results and concurrent biosignal data to estimate a confidence score (e.g., a probability) representing a likelihood (e.g., probability) that an ADHD diagnosis for the user is accurate. For example, the system can: access a series of results corresponding to a cognitive evaluation completed by the user during a test period; access a timeseries of biosignal data recorded during completion of the cognitive evaluation; and access a global model associating results of the cognitive evaluation, biosignal data, and instances of adverse cognitive states in users diagnosed with ADHD. The system can leverage the user's results from the cognitive evaluation and corresponding biosignal data to estimate a confidence score for the user representing a probability of an ADHD diagnosis for the user. If the confidence score exceeds a threshold confidence, the system can interpret an ADHD diagnosis for the user. The system can store this initial ADHD diagnosis and the corresponding confidence score at the ADHD profile of the user.

In one implementation, the system can leverage these cognitive evaluation results and concurrent biosignal data to estimate an intensity of ADHD exhibited by the user. For example, the system can estimate an intensity score for the user based on magnitudes of biosignals recorded during an instance of an identified adverse cognitive state, results of the cognitive evaluation during this instance of the adverse cognitive state, and the global model. In another example, the system can estimate an intensity score for the user based on a duration and/or frequency of the adverse cognitive state, results of the cognitive evaluation during this instance(s) of the adverse cognitive state, and the global model. The system can then store this intensity score at the ADHD profile for the user.

In one implementation, the system can discern between different ADHD types and interpret a more specific diagnosis for the user based on results of the cognitive evaluation, the timeseries of biosignal data, and the global model. For example, the system can leverage magnitudes and patterns observed for particular biosignals to identify a type of ADHD most likely exhibited by the user based on the global model. More specifically, in this example, the system can: interpret an ADHD diagnosis of the attention-deficit type based on identification of a particular trend in a first biosignal linked to the attention-deficit type; interpret an ADHD diagnosis of the hyperactive type based on identification of a particular trend in a second biosignal linked to the hyperactive type; or interpret an ADHD diagnosis of the combination type (e.g., attention-deficit-hyperactive type) based on identification of a particular trend in both the first and second biosignal.

The system can similarly compare biosignal data for the user for multiple biosignals (e.g., GSR, HRV, and STE) to a global ADHD model including each of these biosignals. For example, the system can extract a first timeseries of GSR data corresponding to a particular time period; extract a second timeseries of HRV data corresponding to the particular time period; and extract a third timeseries of STE data corresponding to the particular time period. The system can identify an ADHD type corresponding to these biosignal data based on the global ADHD model and a combination of the first timeseries of GSR data, the second timeseries of HRV data, and the third timeseries of STE data.

### 4.3 User-Specific ADHD Model

The system can access the timeseries of results of the cognitive evaluation to extract insights into the user's cognitive states throughout the cognitive evaluation. The system can isolate sections of the timeseries of results indicating an instance of an adverse cognitive state (e.g., reduced cognitive inhibition) based on the timeseries of results. After isolating instances of the adverse cognitive state, the system can generate a timeseries of ADHD biomarkers for the duration of the cognitive test and then label the timeseries of biosignal data with the timeseries of ADHD biomarkers.

In one implementation, the system can extract a cognitive state change marker from the cognitive evaluation. Generally, the system can: extract a set of ADHD biomarkers, as described above; identify a change from a first cognitive state (e.g., positive) to a second cognitive state (e.g., adverse); and generate a cognitive state change marker for that period of the cognitive evaluation. The system can further isolate cognitive state change markers from different sets of cognitive states. For example, the system can: identify the user changing from a focused cognitive state to a hyperactive cognitive state; identify the user changing from a focused cognitive state to an attention-deficit cognitive state; and generate cognitive state change markers for each type of cognitive state change.

The system can then: synchronize these timeseries of physiological biosignal data and instances of the adverse cognitive state; and implement regression, machine learning, deep learning, and/or other techniques to derive links or correlations between these physiological biosignals and the adverse cognitive state for the user. The system can derive correlations between physiological biosignal data and other cognitive states of the user, such as during a single (e.g., 30 minute) cognitive evaluation or during intermittent testing periods during the user's first day or week wearing the wearable device following the initial cognitive evaluation. The remote computer system can then compile these correlations between physiological biosignal data and cognitive states into an ADHD model unique to the user, such as by compiling these correlations into a new ADHD model for the user or by calibrating an existing generic ADHD model (e.g., the global ADHD model) to align with these correlations.

### 5. Treatment Pathway

Based on the results of the cognitive evaluation and the timeseries of biosignal data recorded during the cognitive evaluation, the system can select a treatment pathway configured to monitor and/or improve behaviors associated with ADHD (e.g., motivation, initiation, attention, emotional regulation). For example, the system can select a treatment pathway including: a recommendation to meet with a mental health provider (e.g., a licensed therapist, a psychiatrist) at a particular frequency; scheduled cognitive exercises configured to evaluate changes in ADHD behaviors exhibited by the user; intervention activities matched to the user and configured to mitigate adverse cognitive events; and/or prompts to the user at a particular frequency soliciting feedback from the user. In one implementation, the system can initially select a generic treatment pathway for the user based on the initial ADHD diagnosis (e.g., type, intensity). Over time, the system can continue evaluating biosignal data and/or cognitive results for the user to modify and/or refine the treatment pathway to converge on a user-specific treatment pathway.

In one implementation, the system can select a treatment pathway for the user based on the ADHD profile of the user. For example, the system can: access a treatment model linking ADHD profiles to ADHD treatment pathways; access the ADHD profile of the user including a confidence score, intensity score, and/or ADHD type corresponding to the ADHD diagnosis of the user; and identify a particular treatment pathway best matched to the user based on the user's ADHD profile and the treatment model.

### 6. Daily Biosignal Tracking

After generating the user-specific ADHD model, the wearable device can: access the set of sensors to record additional biosignal data of the user; access the ADHD model of the user; and—upon detection of biosignal data indicating an instance of an adverse cognitive state based on the ADHD model—alert the user of the instance of the adverse cognitive state to enable the user to be mindful of the user's cognitive state.

In one implementation, the wearable device can detect an adverse cognitive state (e.g., low or high arousal state) and signal the user of the event via the wearable device (e.g., by haptic feedback, emitting a tone, etc.) or via a notification at the mobile device. In another implementation, the companion application can log a series of adverse cognitive states throughout the day and display the list to the user at the end of the day via the user's mobile device. Additionally, the companion application can identify daily trends regarding instances of the adverse cognitive state and inform the user of these trends. The system can also prompt the user at a set time of the day to avoid disrupting the user during the course of the day.

The system can continue recording biosignal data for the user over time to further develop the user-specific ADHD profile. In one implementation, the system can monitor biosignal data for the user each day (e.g., for three days, for one week, for two weeks) to identify patterns and/or trends in the observed biosignal data over the course of the user's day. The system can then store these observed patterns and/or trends at the ADHD profile for the user. For example, the system can record a timeseries of biosignal data at set intervals (e.g., once per minute, once every ten minutes, three times per hour) each day for a set period of time (e.g., one week). In response to detecting a first instance of an adverse cognitive event between 10 AM and 11 AM each day, and a second instance of an adverse cognitive event between 3 PM and 5 PM each day, the system can store these instances of adverse cognitive events in the user-specific ADHD profile. The system can then predict that the user may experience a first instance of an adverse cognitive event each day between 10 AM and 11 AM, and a second instance of an adverse cognitive event each day between 3 PM and 5 PM, and inform the user of this prediction, such that the user may better plan her day around these predicted instances of the adverse cognitive event and/or implement intervention strategies—as discussed below—to mitigate these adverse cognitive events. The system can continue tracking biosignal data over time and refine these observed patterns and/or trends as the system acquires additional data and update and/or modify the user profile as these patterns and/or trends change over time.

Similarly, the system can identify patterns related to other cognitive states exhibited by the user throughout the day, such as a high performance and functionality zone linked to baseline or improved executive function for the user. The system can then leverage these patterns to identify and signal to the user the best conditions (e.g., time of day, environment) for maximum productivity. For example, the system can interpret an adverse cognitive event linked to decreased motivation for the user on most days, approximately four hours after the user wakes up for the day. The system can also interpret an instance of the high performance and functionality zone linked to an increase in motivation (e.g., above average for the user) beginning approximately one hour after the user wakes up and ending as the adverse cognitive event begins. Therefore, the system can prompt the user to complete the most difficult tasks of the day during this instance of the high performance and functionality zone and limit the quantity and/or difficulty of tasks performed during the adverse cognitive event. The system can store this information (e.g., frequency, duration, times of day, patterns) regarding instances of adverse cognitive events and high performance and functionality zones at the ADHD profile for the user.

### 7. Cognitive Exercises

In one implementation, the system can prompt the user to complete a set of cognitive exercises to further refine the ADHD model and update the ADHD profile of the user, in addition to regularly tracking biosignal data for the user. For example, the system can: prompt the user to complete a particular cognitive exercise at least three times, once in the morning, once in the afternoon, and once in the evening (e.g., over a period of one week). The user may notify the system when the user is prepared to initiate the cognitive exercise and the system can record a timeseries of biosignal data throughout the duration of the exercise. If the user completes the exercise efficiently in the morning and in the evening but completes the exercise at a much slower pace in the afternoon, the system can check that the corresponding timeseries of biosignal data reflects this performance based on the ADHD model for the user. Therefore, the system can confirm accuracy of the ADHD model for the user as well as further refine the ADHD model based on this new biosignal data.

Further, the system can serve these cognitive exercises to the user to evaluate the user's progress over time and/or to modify the treatment pathway of the user. For example, the system can prompt the user to complete a series of cognitive exercises over a period of time (e.g., 1 month, 6 months, 1 year). At a first time, the system can prompt the user to complete a first cognitive exercise. The system can then store the results of this first cognitive exercise and/or update the ADHD model of the user as described above based on these results. One month later (e.g., after implementation of a first treatment pathway), the system can prompt the user to complete a second cognitive exercise (e.g., identical to the first cognitive exercise). The system can compare the results of the second cognitive exercise to the results of the first cognitive exercise to evaluate the user's progress in management of her ADHD. If the results of the second cognitive exercise demonstrate an improvement (e.g., a higher overall score) over the results of the first cognitive exercise, the system can confirm a first treatment pathway currently implemented for the user. Alternatively, if the results of the second cognitive exercise demonstrate little to no improvement over the results of the first cognitive exercise, the system can select a second treatment pathway (e.g., a modification of the first treatment pathway, a new treatment pathway) in replacement of the first treatment pathway. Additionally and/or alternatively, the system can serve these results to the user's mental health provider (e.g., the user's psychiatrist) for further evaluation of the current treatment pathway.

### 8. Intervention Activities

In one variation, the system can prompt the user to complete an intervention exercise in response to detecting an instance of the adverse cognitive state. In this variation, the system can: detect an instance of the adverse cognitive state via the wearable device; via the user's mobile device, load an intervention protocol (e.g., a set of intervention activities geared to move past the adverse cognitive state); and prompt the user to complete an intervention activity (e.g., a breathing exercise, a mood diary, cognitive and behavioral exercises, and activities which are tailored to the user, etc.). The system can suggest intervention activities tailored to the user based on information stored at the ADHD profile of the user. The system can suggest different intervention activities based on type (e.g., attention-deficit, hyperactivity), intensity, timing (e.g., time of day) and/or duration of adverse cognitive events.

For example, the system can: access the set of sensors on the wearable device, detect an instance of an adverse cognitive state (e.g., attention-deficit) exhibited by the user; access the ADHD profile of the user to select a first intervention exercise—matched to the user and the adverse cognitive state—based on the treatment pathway defined for the user; signal to the user by vibrating the wearable device; and prompt the user via the mobile device to begin a journaling activity to help regain the user's focus and regulate the adverse cognitive state.

In one implementation, the system can leverage observed patterns and/or trends in biosignal data stored in the user profile to preemptively suggest and/or prompt the user to complete intervention activities leading up to or at a start of predicted adverse cognitive events. For example, the system can: access the user profile and identify a predicted adverse cognitive event at a particular time of day; load an intervention protocol stored in the user profile; and select a particular intervention activity matched to the predicted adverse cognitive event for this user. Then, ten minutes before the predicted adverse cognitive event, the system can alert the user of the predicted adverse cognitive event and prompt the user to complete the particular intervention activity at the particular time of day.

The system can monitor whether intervention activities are successful for the specific user based on biosignal data recorded during completion of these intervention activities and/or based on feedback from the user and/or a third-party user (e.g., the user's licensed therapist). If a particular intervention activity is highly successful at mitigating instances of the adverse cognitive state for the user than other intervention activities (e.g., based on recorded biosignal data and/or feedback from the user), the system can assign a high rank to this particular intervention activity. Alternatively, if a particular intervention activity is less successful at mitigating instances of the adverse cognitive state for the user, the system can assign a low rank to this particular intervention activity. Additionally and/or alternatively, the system can remove this low-ranked intervention activity from the intervention protocol for this user. Therefore, over time, the system can learn which intervention activities work best for the user to mitigate instances of adverse cognitive states and notify the user of these intervention activities such that the user may be better equipped to handle adverse cognitive events and mitigate interference of these events with user performance and everyday life.

In one implementation, the wearable device can monitor biosignals of the user leading up to completion of the intervention activity and continue to monitor biosignals of the user after completion of the intervention activity. The system can leverage biosignal data recorded before, during, and after completion of these intervention exercises to interpret which activities are most effective for the user. For example, the system can record an effectiveness value for each intervention activity performed based on changes in magnitudes of biosignal data collected before, during, and after each intervention activity. The system can then rank each intervention activity according to the effectiveness values.

### 9. User Feedback

The system can prompt the user to periodically confirm instances of adverse cognitive states and/or high performance and functionality zones to further refine the user-specific ADHD model and update the ADHD profile for the user. For example, at the end of a particular day, in response to detecting a severe (e.g., high intensity, long duration) instance of an adverse cognitive state during the day, the system can prompt the user to record a brief journal entry detailing the instance of the adverse cognitive state including: whether the user felt productive; whether the user felt distracted; whether the user felt successful; a quantity of work the user completed; a quality of work the user completed; a type of activity the user completed; whether the user performed an intervention activity before, during, and/or after the instance of the adverse cognitive state; etc. The system can similarly gather information from the user regarding instances of high performance and functionality zones (e.g., high productivity). The system can then leverage this information entered by the user to: confirm or reject instances of the adverse cognitive state; identify false-negative and/or false-positive instances of the adverse cognitive state; update the ADHD model for the user; update the ADHD profile of the user; update the treatment pathway selected for the user; and/or select intervention activities for the user better matched to the user.

The system can similarly prompt the user to periodically enter environmental characteristics corresponding to instances of both adverse cognitive states and high performance and functionality zones. For example, in response to detecting an instance of an adverse cognitive state, the system can prompt the user to record a brief journal entry detailing environmental characteristics surrounding the instance of the adverse cognitive state, including: a location of the user; whether the user is alone, with other people, or in a crowded location; a noise level of the location; whether the user is listening to music; etc. The system can store this feedback from the user at the ADHD profile for the user. The system can then leverage this feedback to suggest particular environmental conditions to the user to mitigate future instances of the adverse cognitive state.

### 10. Third-Party Feedback

In one implementation, the companion application can prompt the user to share all or part of her user profile with a third-party user (e.g., the user's licensed therapist) running the companion application on a different device such that the system can update the other user with certain data about the user (e.g., adverse cognitive states and trends). For example, the system can: generate a user profile including a user-specific ADHD model for tracking an adverse cognitive state (e.g., low attentional control) that the user would like to discuss during a therapy session with a licensed therapist; track instances of the adverse cognitive state for a particular period of time (e.g., a week); prompt the user to share a list of instances of the adverse cognitive state from the particular period of time with her licensed therapist; and—upon receiving instructions from the user to share a list of instances of the adverse cognitive state—send the list of instances of the adverse cognitive state to the licensed therapist's device running the companion application. The licensed therapist may discuss these instances of the adverse cognitive state with the user to extract further details regarding severity of these adverse cognitive states and/or to discern false-positive and false-negative adverse cognitive states. The licensed therapist may then leverage these details to generate feedback (e.g., via labelled biosignal data) from this therapy session for uploading to the system. Based on this feedback from the user's therapist, the system can update the ADHD model for the user to more accurately detect adverse cognitive events in the future.

The system can enable the third-party user to implement and/or suggest therapeutic techniques (e.g., behavioral therapy, medication) matched to the ADHD profile of the user. For example, rather than initially implementing strategies for treating a generic ADHD diagnosis, the system can enable the user's therapist to suggest a medication linked to a particular type of ADHD and at a dosage associated with an intensity identified in the user's ADHD profile.

### 11. Evaluating Progress Over Time

The system can continue monitoring biosignal data of the user over time to continue refining the ADHD model for the user, updating the user's ADHD profile, and suggesting updated treatment pathways, intervention activities, and general tools for mitigating negative effects of ADHD best matched to the ADHD profile of the user.

As the system collects additional biosignal data from the user—in combination with feedback from the user and/or the user's licensed therapist—the system can converge on a refined user-specific ADHD model. For example, the system can: monitor the user's biosignals on a daily basis; serve the user a series of cognitive exercises over a period of time (e.g., one month, one year); collect feedback from the user's licensed therapist over the period of time; collect feedback from the user over the period of time; serve and/or inform the user of intervention activities for completion during adverse cognitive events; etc. Based on all of this information, the system can estimate an updated confidence score in the user's ADHD diagnosis and adjust accordingly. If the confidence score is above a threshold confidence (e.g., greater than the original confidence score), the system can affirm the user's ADHD diagnosis. If however, the confidence score is below the threshold confidence, the system can elect to reevaluate and/or refine the user's ADHD diagnosis and/or cooperate with the user's mental health provider to refine the user's ADHD diagnosis.

Further, based on all of this information collected over time (e.g., biosignal data, feedback from the user and/or the user's licensed therapist, results of cognitive exercises), the system can monitor and/or track the user's progress in management of her ADHD diagnosis. The system can inform the user and/or the user's mental health provider of this progress and leverage this information to modify and/or select treatment pathways. For example, over a six month period, the system can identify an increase in overall user productivity, such as based frequency of adverse cognitive events detected from biosignal data, intensity of biosignal data during these adverse cognitive events, feedback from the user and her therapist, and/or results of cognitive exercises. The system can therefore leverage this data to reaffirm the user's current treatment pathway. Alternatively, if the system identifies a decrease in quality of work performed (e.g., based on results of cognitive exercises) in addition to the increase in overall user productivity, the system can select a second treatment pathway (e.g., a modification of the first treatment pathway) configured to increase the quality of the user's work while maintaining the increase in overall user productivity.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

1. A method of characterizing ADHD for a user comprising:
during a first time period:
accessing a series of results of a cognitive evaluation executed by the user during a test period within the first time period;
accessing a first timeseries of biosignal data collected via a set of sensors integrated into a wearable device worn by the user during the first time period;
accessing a global model linking results of the cognitive evaluations and biosignal data to cognitive states of users diagnosed with ADHD;
interpreting a first confidence score for the user based on the series of results of the cognitive evaluation, the first timeseries of biosignal data, and the global model, the first confidence score representing a probability of a positive ADHD diagnosis for the user; and
in response to the first confidence score exceeding a threshold confidence:
selecting a first treatment pathway for the user based on the series of results of the cognitive evaluation and the first timeseries of biosignal data; and
deriving an ADHD model for the user linking biosignal data recorded for the user to instances of cognitive states exhibited by the user based on the series of results of the cognitive evaluation, the first timeseries of biosignal data, and the global model; and
during a second time period succeeding the first time period:
generating a first prompt to complete a cognitive exercise based on the first treatment pathway selected for the user;
transmitting the first prompt to the user;
accessing a second timeseries of biosignal data collected via the set of sensors integrated into the wearable device worn by the user during execution of the cognitive exercise;
characterizing user progress based on results of the cognitive exercise, the second timeseries of biosignal data, and the ADHD model; and
in response to user progress exceeding a threshold progress, confirming selection of the first treatment pathway for the user.

2. The method of claim 1, further comprising:
interpreting a second confidence score for the user based on results of the cognitive exercise, the second timeseries of biosignal data, and the ADHD model; and
in response to the second confidence score exceeding the threshold confidence, affirming the positive ADHD diagnosis for the user.

3. The method of claim 1, further comprising, in response to user progress falling below the threshold progress, selecting a second treatment pathway in replacement of the first treatment pathway based on the ADHD model, the results of the cognitive exercise, and the second timeseries of biosignal data.

4. The method of claim 1, further comprising, during a third time period succeeding the first time period:
accessing a third timeseries of biosignal data collected via the set of sensors integrated into the wearable device worn by the user during the third time period;
interpreting a series of cognitive states of the user during the third time period based on the third timeseries of biosignal data and the ADHD model; and
in response to a first cognitive state, in the series of cognitive states, corresponding to an adverse cognitive state:
generating a second prompt to execute an intervention exercise based on the adverse cognitive state and the first treatment pathway; and
transmitting the second prompt to the user.

5. The method of claim 4:
wherein accessing the third timeseries of biosignal data collected via the set of sensors integrated into the wearable device comprises:
accessing a timeseries of skin conductance data, in the third timeseries of biosignal data, recorded by an electrodermal activity sensor, in the set of sensors, on the wearable device; and
accessing a timeseries of pulse data, in the third timeseries of biosignal data, recorded by a heart rate sensor, in the set of sensors, on the wearable device; and
wherein interpreting the series of cognitive states of the user based on the third timeseries of biosignal data and the ADHD model comprises interpreting the series of cognitive states of the user based on the timeseries of skin conductance data, the timeseries of pulse data, and the ADHD model.

6. The method of claim 5:
further comprising, during the third time period, accessing a timeseries of environmental data comprising:
accessing a timeseries of air humidity data recorded by an ambient humidity sensor on the wearable device; and
accessing a timeseries of air temperature data recorded by an ambient temperature sensor on the wearable device; and
wherein interpreting the series of cognitive states of the user during the third time period based on the third timeseries of biosignal data and the ADHD model comprises interpreting the series of cognitive states of the user during the third time period based on the third timeseries of biosignal data, the timeseries of environmental data, and the ADHD model.

7. The method of claim 1, wherein accessing the first timeseries of biosignal data collected via the set of sensors integrated into the wearable device worn by the user during the first time period comprises:
accessing a timeseries of skin conductance data recorded by an electrodermal activity sensor on the wearable device;
accessing a timeseries of pulse data recorded by a heart rate sensor on the wearable device;
accessing a timeseries of relative humidity data recorded by an ambient humidity sensor on the wearable device; and
accessing a timeseries of temperature data recorded by an ambient temperature sensor on the wearable device.

8. The method of claim 1, wherein deriving the ADHD model for the user comprises:
linking a first segment of the first timeseries of biosignal data to a first portion of the cognitive evaluation configured to evaluate attentional control of the user;
linking a second segment of the first timeseries of biosignal data to a second portion of the cognitive evaluation configured to evaluate cognitive inhibition of the user;
linking a third segment of the first timeseries of biosignal data to a third portion of the cognitive evaluation configured to evaluate working memory of the user;
generating a set of ADHD biomarkers in the first timeseries of biosignal data corresponding to portions of the cognitive evaluation based on the global model; and
deriving the ADHD model for the user based on the set of ADHD biomarkers.

9. The method of claim 8, further comprising:
interpreting a series of cognitive states of the user during the first time period based on the first timeseries of biosignal data and the set of ADHD biomarkers; and
in response to interpreting a transition from a positive cognitive state in the series of cognitive states, to an adverse cognitive state, in the series of cognitive states, generating a cognitive state change marker at a transition period corresponding to the transition.

10. The method of claim 1:
further comprising, during the first time period:
interpreting an adverse cognitive state for the user during the first time period based on the first timeseries of biosignal data and the ADHD model;
recording a first duration of the adverse cognitive state;
recording a first frequency of the adverse cognitive state of the user during the test period; and
estimating an ADHD intensity score for the user based on the first duration, the first frequency, the series of results of the cognitive evaluation, and the global model;
further comprising, during the first time period, generating an ADHD profile for the user containing the intensity score and the first confidence score; and
wherein selecting the first treatment pathway based on the series of results of the cognitive evaluation and the first timeseries of biosignal data comprises selecting the first treatment pathway based on the ADHD profile of the user.

11. The method of claim 1, wherein deriving the ADHD model for the user linking biosignal data recorded for the user to instances of cognitive states exhibited by the user based on the series of results of the cognitive evaluation, the first timeseries of biosignal data, and the global model comprises interpreting an ADHD diagnosis along a spectrum defining:
the first confidence score representing a probability of a positive ADHD diagnosis for the user;
an ADHD type; and
an intensity score representing intensity of ADHD exhibited by the user.

12. The method of claim 1, wherein selecting the first treatment pathway matched to the user based on the series of results of the cognitive evaluation and the first timeseries of biosignal data comprises:
generating an ADHD profile for the user based on results of the cognitive evaluation, the first timeseries of biosignal data, and the global model, the ADHD profile comprising:
the first confidence score;
an intensity score representing intensity of ADHD exhibited by the user; and
an ADHD type exhibited by the user;
accessing a treatment model linking ADHD profiles of users diagnosed with ADHD to ADHD treatment pathways; and
identifying a particular treatment pathway matched to the user based on the ADHD profile of the user and the treatment model.

13. The method of claim 1, wherein interpreting the first confidence score for the user based on the series of results of the cognitive evaluation, the first timeseries of biosignal data, and the global model comprises characterizing a correlation between the first timeseries of biosignal data and biosignal data of users diagnosed with ADHD based on the global model.

14. The method of claim 1, wherein deriving the ADHD model for the user linking biosignal data recorded for the user to instances of cognitive states exhibited by the user based on the series of results of the cognitive evaluation, the first timeseries of biosignal data, and the global model comprises:
interpreting an attention-deficit ADHD type based on identification of a first particular trend in a first biosignal of the first timeseries of biosignal data linked to the attention-deficit type;
interpreting a hyperactive ADHD type based on identification of a second particular trend in a second biosignal of the first timeseries of biosignal data linked to the hyperactive type; and
interpreting an ADHD diagnosis of a combination type based on identification of the first particular trend and the second particular trend.

15. The method of claim 1, wherein deriving the ADHD model for the user linking biosignal data recorded for the user to instances of cognitive states exhibited by the user based on the series of results of the cognitive evaluation, the first timeseries of biosignal data, and the global model comprises:
extracting a first timeseries of galvanic skin response data from the first timeseries of biosignal data corresponding to a particular time period during the first time period;
extracting a second timeseries of heart rate variability data from the first timeseries of biosignal data corresponding to the particular time period;
extracting a third timeseries of STE data from the first timeseries of biosignal data corresponding to the particular time period; and
identifying an ADHD type based on the global ADHD model and a combination of the first timeseries of galvanic skin response data, the second timeseries of heart rate variability data, and the third timeseries of STE data.

16. A method of characterizing ADHD for a user comprising:
during a first time period:
accessing a series of results of a cognitive evaluation executed by the user during a test period within the first time period;
accessing a first timeseries of biosignal data collected via a set of sensors integrated into a wearable device worn by the user during the first time period;
accessing a global model linking results of the cognitive evaluation and biosignal data to cognitive states of users diagnosed with ADHD; and
deriving an ADHD model for the user linking biosignal data recorded for the user to instances of cognitive states exhibited by the user based on the series of results of the cognitive evaluation, the first timeseries of biosignal data, and the global model; and
during a second time period succeeding the first time period:
accessing a second timeseries of biosignal data collected via the set of sensors integrated into the wearable device worn by the user during the second time period;
interpreting a series of cognitive states of the user during the second time period based on the second timeseries of biosignal data and the ADHD model; and
at a first time during the second time period, in response to a first cognitive state, in the series of cognitive states, corresponding to an adverse cognitive state:
generating a prompt to execute an intervention exercise associated with the first treatment pathway; and
transmitting the prompt to a mobile device associated with the first user.

17. The method of claim 16:
wherein tracking the second timeseries of biosignal data collected via the set of sensors integrated into the wearable device worn by the user during the third time period comprises:
tracking a timeseries of skin conductance data recorded by an electrodermal activity sensor on the wearable device; and
tracking a timeseries of pulse data recorded by a heart rate sensor on the wearable device; and
wherein interpreting the series of cognitive states of the user based on the second timeseries of biosignal data and the ADHD model comprises interpreting the cognitive state of the user based on the timeseries of skin conductance data, the timeseries of pulse data, and the ADHD model.

18. The method of claim 16:
further comprising, during the second time period, tracking a timeseries of environmental data comprising:
accessing a first subset of timeseries of environmental data representing relative humidity of air around the user recorded by an ambient humidity sensor on the wearable device; and
accessing a second subset of timeseries of environmental data representing relative heat of air surrounding the user recorded by an ambient temperature sensor on the wearable device; and
wherein interpreting the series of cognitive states of the user based on the second timeseries of biosignal data and the ADHD model comprises interpreting a cognitive state of the user based on the second timeseries of biosignal data, the timeseries of environmental data, and the ADHD model.

19. The method of claim 16, further comprising, in response to expiration of the second time period:
generating a report comprising a summary of adverse cognitive states exhibited by the user during the second period of time based on the series of cognitive states;
transmitting the report to a first device associated with the user; and
transmitting the report to a health care provider associated with the user.

20. The method of claim 16, further comprising, during a third time period succeeding the second time period:
accessing a third timeseries of biosignal data collected via the set of sensors integrated into the wearable device worn by the user during the third time period;
interpreting a second series of cognitive states of the user during the third time period based on the third timeseries of biosignal data and the ADHD model;
predicting an instance of the adverse cognitive state during a fourth time period succeeding the third time period based on the first series of cognitive states and the second series of cognitive states;
generating a notification indicating prediction of the instance of the adverse cognitive state during the fourth time period; and
transmitting the notification to the user.
